(19) United States
(12) Patent Application Publication
(10) Pub. No.: US 2017/0119556 A1
(43) Pub. Date: May 4, 2017

(54) IMPLANTABLE MEDICAL DEVICE WITH BONDING REGION

(71) Applicant: BOSTON SCIENTIFIC SCIMED, INC., Maple Grove, MN (US)

(72) Inventors: Thomas Holly, Galway (IE); Martyn G. Folan, Galway (IE); Thomas M. Keating, Galway (IE); Michael Walsh, Galway (IE)

(21) Appl. No.: 15/335,566

(22) Filed: Oct. 27, 2016

Related U.S. Application Data
(60) Provisional application No. 62/248,413, filed on Oct. 30, 2015.

(51) Int. Cl. A61F 2/82; D04B 21/12

(52) U.S. Cl. CPC A61F 2/82; D04B 21/12; A61F 2230/0069

(57) ABSTRACT

Medical devices and methods for making and using a medical device are disclosed. An example medical device may include an implantable endoprosthesis. The implantable endoprosthesis may include a cylindrical body having a proximal end, a distal end, and an axial bonding region extending between the proximal end and the distal end. The cylindrical body may include one or more winding filaments and a plurality of discrete axial bonds disposed along the axial bonding region. The discrete axial bonds may secure together edge regions of the one or more winding filaments.

### CROSS-REFERENCE TO RELATED APPLICATIONS

This application claims priority under 35 U.S.C. §119 to U.S. Provisional Application Ser. No. 62/248,413, filed Oct. 30, 2015, the entirety of which is incorporated herein by reference.

### TECHNICAL FIELD

The present disclosure pertains to medical devices, and methods for manufacturing medical devices. More particularly, the present disclosure pertains to implantable medical devices.

### BACKGROUND

A wide variety of intracorporeal medical devices have been developed for medical use, for example, intravascular use. Some of these devices include guidewires, catheters, stents, and the like. These devices are manufactured by any one of a variety of different manufacturing methods and may be used according to any one of a variety of methods.

### SUMMARY

This disclosure provides design, material, manufacturing method, and use alternatives for medical devices. An example medical device may include an implantable endoprosthesis comprising a cylindrical body having a proximal end, a distal end, and an axial bonding region extending between the proximal end and the distal end; wherein the cylindrical body includes one or more winding filaments; and a plurality of discrete axial bonds disposed along the axial bonding region, the discrete axial bonds securing together edge regions of the one or more winding filaments.

Alternatively or additionally to any of the embodiments above, wherein the one or more winding filaments includes a braided portion, a knitted portion, or both.

Alternatively or additionally to any of the embodiments above, wherein the one or more winding filaments includes a braided portion and a knitted portion, wherein the braided portion is interwoven with the knitted portion.

Alternatively or additionally to any of the embodiments above, wherein the winding filaments includes a first filament having a first radial compression strength and a second filament having a second radial compression strength different from the first radial compression strength.

Alternatively or additionally to any of the embodiments above, wherein the discrete axial bonds include a weld.

Alternatively or additionally to any of the embodiments above, wherein the cylindrical body further comprises a bifurcated portion.

Alternatively or additionally to any of the embodiments above, wherein the cylindrical body includes a second axial bonding region.

Another example implantable endoprosthesis comprises a tubular scaffold including a proximal end, a distal end and a longitudinal axis, the tubular scaffold including at least a first filament including a first set of windings and a second set of windings; a bonding region extending along the tubular scaffold including a plurality of discrete bonds; wherein the one or more discrete bonds secure the first set of windings to the second set of windings.

Alternatively or additionally to any of the embodiments above, further comprising a second filament, wherein the first filament includes a braided portion and the second filament includes a knitted portion.

Alternatively or additionally to any of the embodiments above, wherein the braided portion and the knitted portion are interwoven.

Alternatively or additionally to any of the embodiments above, further comprising a second filament, wherein the first filament includes a first material and the second filament includes a second material different from the first material.

Alternatively or additionally to any of the embodiments above, wherein the tubular scaffold includes a bifurcated portion.

Alternatively or additionally to any of the embodiments above, wherein the tubular scaffold includes a second bonding region including a plurality of discrete bonds along the bifurcated portion.

An example method of making an implantable endoprosthesis comprises positioning at least one filament on along a planar surface of a base, the base including a plurality of projections extending away from the surface; wherein positioning the at least one filament on the planar surface of the base includes winding the at least one filament along the base by winding the filament about the plurality of projections to form a substantially planar stent structure, the planar stent structure including a first side and a second side and one or more interstices therebetween; removing the planar stent structure from the planar surface; positioning the planar stent structure around a shaping mandrel; and attaching the first side of the stent structure to the second side of the stent structure.

Alternatively or additionally to any of the embodiments above, wherein attaching the first side of the stent structure to the second side of the stent structure further includes forming a bonding region.

Alternatively or additionally to any of the embodiments above, wherein the bonding region includes at least one weld.

Alternatively or additionally to any of the embodiments above, wherein positioning the at least one filament on a planar surface comprises both braiding and knitting the filament around the plurality of projections.

Alternatively or additionally to any of the embodiments above, wherein positioning the planar stent structure around a shaping mandrel includes positioning the planar stent structure around a bifurcated mandrel.

Alternatively or additionally to any of the embodiments above, wherein positioning the at least one filament between at least two of the plurality of projections to form a substantially planar stent structure includes forming a third side and a fourth side.

Alternatively or additionally to any of the embodiments above, further comprising attaching the third side to the fourth side to form a second bonding region.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example implantable medical device;

FIG. 2 illustrates a perspective view of an example base including outwardly extending projections;

FIG. 3 illustrates a top view of an example base including outwardly extending projections;

FIG. 4 illustrates an example base having at least one filament positioned thereon;

FIG. 5 illustrates an example planar stent structure;

FIG. 6 illustrates an example stent structure positioned on a mandrel;

FIG. 7 illustrates an example stent structure being removed from a mandrel;

FIG. 8 illustrates an example multi-filament stent pattern;

FIG. 9 illustrates an example base having at least one filament positioned thereon;

FIG. 10 illustrates an example mandrel;

FIG. 11 illustrates an example bifurcated stent.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the disclosure.

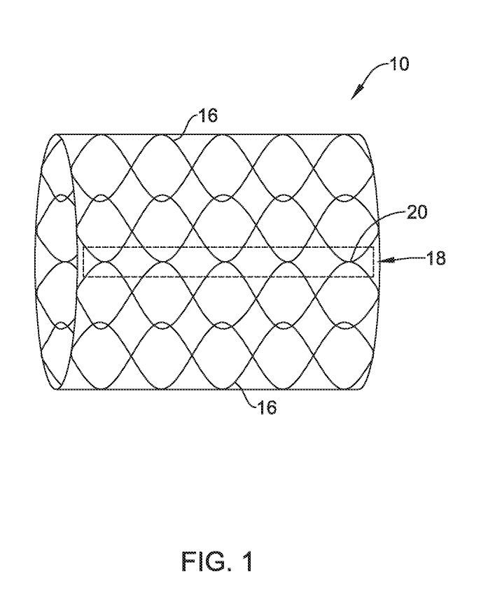

FIG. 1 illustrates an example implantable medical device 10. Implantable medical device 10 may be configured to be positioned in a body lumen for a variety of medical applications. For example, implantable medical device 10 may be used to treat a stenosis in a blood vessel, used to maintain a fluid opening or pathway in the vascular, urinary, biliary, tracheobronchial, esophageal, or renal tracts, or position a device such as an artificial valve or filter within a body lumen, in some instances. In some instances, implantable medical device 10 may be a prosthetic graft, a stent-graft, or a stent (e.g., a vascular stent, tracheal stent, bronchial stent, esophageal stent, etc.), an aortic valve, filter, etc. Although illustrated as a stent, implantable medical device 10 may be any of a number of devices that may be introduced endoscopically, subcutaneously, percutaneously or surgically to be positioned within an organ, tissue, or lumen, such as a heart, artery, vein, urethra, esophagus, trachea, bronchus, bile duct, or the like.

Implantable medical device 10 may include one or more different design configurations and/or components. For example, medical device 10 may have an expandable tubular framework with open ends and defining a lumen therethrough. In some instances medical device 10 may be a self-expanding stent. Self-expanding stent examples may include stents having one or more filaments 16 combined to form a rigid and/or semi-rigid stent structure. Further, wires 16 may be a solid member of a round or to non-round cross-section or may be tubular (e.g., with a round or non-round cross-sectional outer surface and/or round or non-round cross-sectional inner surface).

Medical device (e.g., stent) 10 may be designed to shift between a first or “unexpanded” configuration and a second or “expanded” configuration. In at least some instances, stent 10 may be formed from a shape memory material (e.g., a nickel-titanium alloy such as nitinol) that can be constrained in the unexpanded configuration, such as within a delivery sheath, during delivery and that self-expands to the expanded configuration when unconstrained, such as when deployed from a delivery sheath and/or when exposed to a pre-determined temperature conditions to facilitate expansion. The precise material composition of stent 10 can vary, as desired, and may include the materials disclosed herein.

In some circumstances, it may be desirable to customize medical device 10 to address particular medical applications. Further, in some instances it may be desirable to configure medical device 10 to include one or more filaments interwoven in a particular arrangement. For example, some implantable stents may include an open, mesh-like configuration. In some instances, the open, mesh-like configuration may resemble a braided, knitted and/or woven stent structure. In other words, one or more stent filaments 16 may be braided, intertwined, interwoven, weaved, knitted or the like to form the stent structure 10.

As stated above and will be discussed in greater detail below, the stent structure 10 may be constructed from one or more different braiding, weaving, knitting or similar techniques to form a single stent structure 10. Furthermore, different portions of stent structure 10 may include varying mechanical properties corresponding to different stent structures (e.g., portions of stent 10 having differing design configurations). For example, a portion of stent 10 including a braided portion may exhibit different radial compression strength as compared to a portion of the stent 10 having a knitted or woven structure. For purposes of this disclosure, a “braided” stent structure may be defined as one or more interwoven wires that are weaved together such that the wires may be easily compressed, yet easily return (e.g., “spring back”) to a pre-compressed shape. In contrast, for purposes of this disclosure, a “knitted” stent structure may be defined as one or more interlocking wires that are combined into one or more interlocking loops that may be interdependent on one another. In other words, a “knitted” structure may include interlocking loops that work together to create a stent structure having greater compressive strength as compared a braided stent structure, for example. Further, it is contemplated that other mechanical and/or physical stent properties may be vary in accordance with different stent designs, materials and/or manufacturing techniques.

Some stent structures are contemplated that include only braided filaments. Some stent structures are contemplated that only include knitted filaments. Furthermore some stent structures are contemplated that include one section with braided filaments and another section with knitted filaments. In such instances, the pattern and/or arrangement of the different sections can vary. For example, a stent structure may have braided filaments along a first portion (e.g., a first “half”) and may have a knitted filaments along a second portion (e.g., a second “half”). These are just examples.

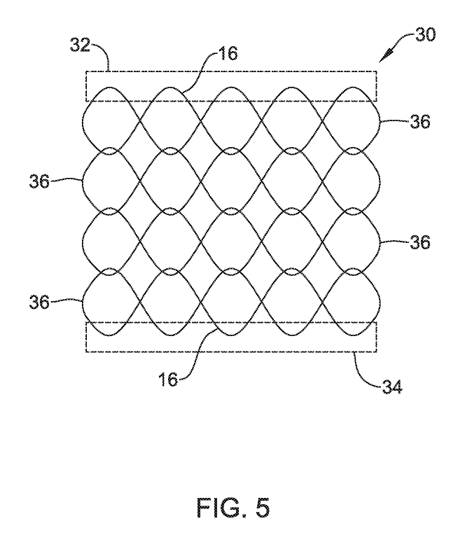

As will be discussed in greater detail below, FIG. 1 shows stent 10 including a bonding region 18. Bonding region 18 may extend along the longitudinal axis of stent 10. Bonding region 18 may include one or more bonds 20. In some instances, bonds 20 may be defined as the attachment and/or combination of one or more end regions 36 (shown in FIG. 5) of wires 16. While FIG. 1 depicts bonds 20 as being longitudinally aligned along the longitudinal axis of stent 10, it is contemplated that bonds 20 may be distributed along stent 10 in a variety of patterns and/or configurations.

While the stent 10 shown in FIG. 1 is depicted as being generally cylindrical in shape and including a substantially uniform pattern and/or distribution of filaments and/or wires 16, it is contemplated that in some instances it may be desirable to construct stent 10 using more complicated or intricate stent patterns, configurations or structural geometries. For example, in some instances it may be desirable to utilize one or more assembly techniques (e.g., braiding and/or knitting) to construct a variety of different stent scaffolds.

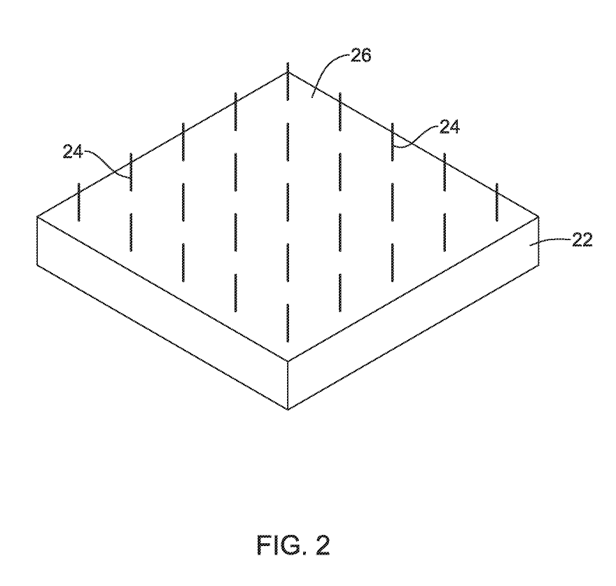

To that end, FIG. 2 illustrates an example base member 22 having an outer surface 26. Base member 22 may be defined as a substantially and/or at least partially planar (e.g., substantially flat) structure. While depicted as a square in FIG. 2, it is contemplated that base member 22 may be any shape. For example, base member may be circular, rectangular, ovular, triangular or the like.

FIG. 2 show projections 24 extending away from surface 26 of base member 22. In some instances, projections 24 may resemble pegs, pins, screws and/or rods extending away from base member 22. However, this is not intended to be limiting. For example, it is contemplated that projections 24 may be a variety of shapes and extend away at any angle with respect to surface 26. For example, in some examples slotted grooves may be utilized perform the methods disclosed herein.

Further, while FIG. 2 shows twenty-five projections 24 arranged in a grid-like pattern, it is contemplated that more or fewer projections 24 may be utilized in conjunction with base 22. For example, base 22 may include 2, 3, 4, 5, 6, 7, 8, 9, 10, 15, 20, 30, 40 50, 100 or more projections arranged in a variety of patterns and/or distributions along base 22. In some instances, the arrangement of projections 24 may be determined by a particular stent geometry and/or design configuration.

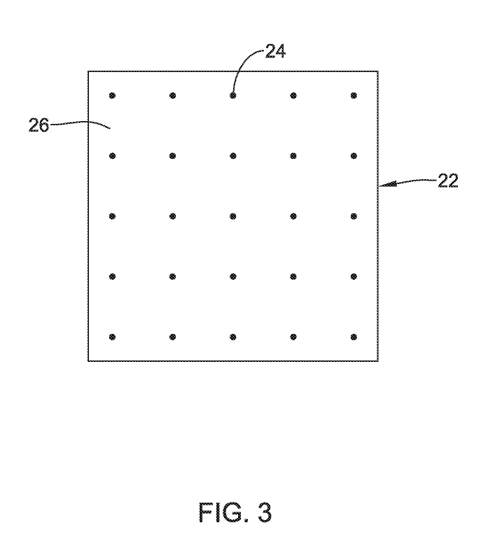

Base 22 (including projections 24) may be utilized to construct a planar (e.g., flat) stent structure. The planar stent structure may subsequently be formed into a variety of three-dimensional stent configurations (discussed below). FIG. 3 shows a top view of the base 22 and projections 24 illustrated in FIG. 2. As discussed above, it can be appreciated that projections 24 may be configured in a variety of patterns, designs, arrangements, distributions, etc. along base 22.

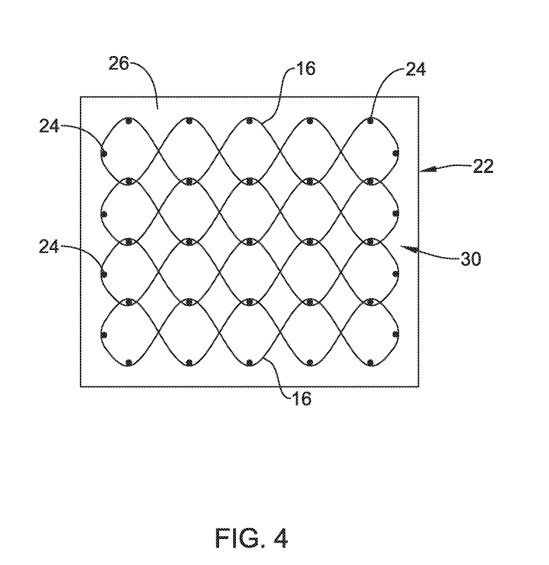

FIG. 4 shows an example filament 16 positioned (e.g., wound, wrapped) around projections 24 of base 22 to form a planar stent structure 30 (shown in FIG. 5 as removed from base 22 of FIG. 4). The pattern illustrated in FIG. 4 is merely an example. It is contemplated that filament 16 may be wound around projections 24 in a variety of different configurations.

Furthermore, it is contemplated that more than one filament 16 may be utilized in the construction of planar stent structure 30. For example, 2, 3, 4, 5, 6, 7, 8, 9, 10, 15, 20, 30, 40, 50 or more filaments 16 may be utilized to form stent structure 30. Additionally, as described above, stent structure 30 may be constructed using one of more different techniques to combine wires 16. For example, one or more portions of planar stent structure 30 may be formed by braiding one or more filaments 16. Additionally, one or more portions of planar stent structure 30 may be formed by knitting one or more filaments 16. While some planar stent structures 30 may be formed using a single technique (e.g., braiding, knitting, weaving, etc.), it is contemplated that more than one technique may be utilized together within the same planar stent structure 30. For example, in some instances one or more wires 6 may be interlocked (via a knitting technique, for example) with one or more wire 16 which are interwoven together (via a braiding technique, for example). While the above examples discusses knitting and braiding as two construction techniques, it is contemplated that planar stent structure 30 may be formed using any stent construction techniques that interweave, interlock, combine, blend, twist, link, intertwine, etc. one or more stent filaments 16.

As stated above, FIG. 5 shows the planar stent structure 30 after being removed from the base 22 shown in FIG. 4. As shown in FIG. 5, planar stent structure 30 may include a first set of stent windings 32 (illustrated in FIG. 5 by a first dashed box) and a second set of windings 34 (illustrated in FIG. 5 by a second dashed box). Further, FIG. 5 illustrates that each set of windings 32/34 include edge regions 36 corresponding to various loops included in planar stent structure 30.

In some examples, prior to being removed from base member 22, additional processing may be applied to stent structure 30 (while on base 22). For example, an annealing process may be applied to stent structure 30 while wound along projections 24 of base member 22 (shown in FIG. 4). In some examples the annealing process may be a low-temperature anneal. The annealing process may “heat set” stent structure 30 such that when stent structure 30 is removed from base member 22, stent structure 30 substantially retains its planar form.

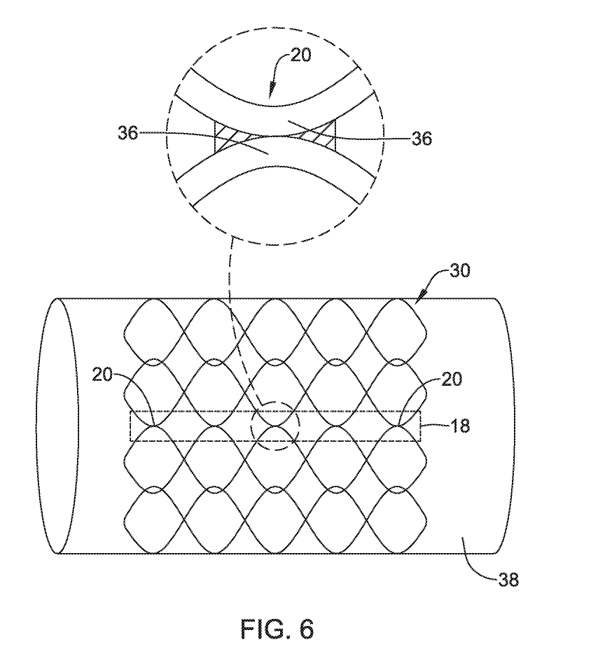

In some instances it may be desirable to transform the planar stent structure 30 shown in FIG. 5 into a three-dimensional stent structure designed to treat a target area in the body. FIG. 6 shows the planar stent structure 30 of FIG. 5 positioned along (e.g., wrapped around) an example shaping mandrel 38. FIG. 6 illustrates the axial bonding region 18 (described above with respect to FIG. 1) including bonds 20.

It can be appreciated the bonding region 18 shown in FIG. 6 defines the combination and/or attachment of the first set of windings 32 with the second set of windings 34 shown in FIG. 5. Further, the detailed view of FIG. 6 shows that in some examples, edge regions 36 (corresponding to the loop portions of stent structure 30) may be combined to attach the first set of windings 32 to the second set of windings 34.

The edge regions 36 of windings 32/34 may be combined using a variety of methodologies. For example, in some instances edge regions 36 may be attached to another via welding. However, this is just an example. It is contemplated that edge regions may be attached to one another using similar bonding techniques such as gluing, tacking, brazing, soldering, or the like. As stated above, the detailed view of FIG. 6 shows edge regions 36 of example windings 32/34 being combined and/or attached. However, even though not shown in the detailed view, it is contemplated that the edge regions 36 may be combined (e.g., melted) together to form a singular structure (e.g., a monolithic stent filament and/or stent strut).

It can be appreciated the positioning (e.g., wrapping) stent structure 30 around shaping mandrel 38 may form planar stent structure 30 into the shape of shaping mandrel 38. Therefore, it can further be appreciated that a variety of different shaping mandrel designs may be utilized to construct three-dimensional stents having a variety of different shapes. For example, as will be discussed further below, shaping mandrel 38 may include one or more extensions or legs (e,g., a bifurcated shape) designed to treat particular vessel geometries in the body.

The above discussion describes a stent manufacturing methodology that initially forms a planar stent structure 30 on a planar base member 22 and later shapes that planar stent structure 30 into a particular three-dimensional stent structure 10 using a shaped mandrel 38. It should be appreciated that this methodology may be utilized to form stent configurations (e.g., self-expanding stent configurations) that are more intricate that those formed from existing manufacturing methods. For example, by winding filaments 16 along planar base 22 before forming the three-dimensional stent structure 10, one or more different manufacturing techniques (such as braiding and knitting) may be combined to yield a single stent structure having a multitude of different arrangements, patterns, structures, and/or distributions that may otherwise be difficult to construct using existing methods.

Furthermore, as stated above, the ability to utilize different manufacturing techniques (e.g., braiding, knitting, etc.) may allow stent 10 to be tailored to have different physical properties in different portions of the stent structure. For example, portions of the stent including a particular stent manufacturing method may have a radial strength that differs from another portion of the stent formed from a different manufacturing methodology. Other physical properties may be customized using similar techniques (e.g., combing braided with knitted portions within the same stent structure, etc.).

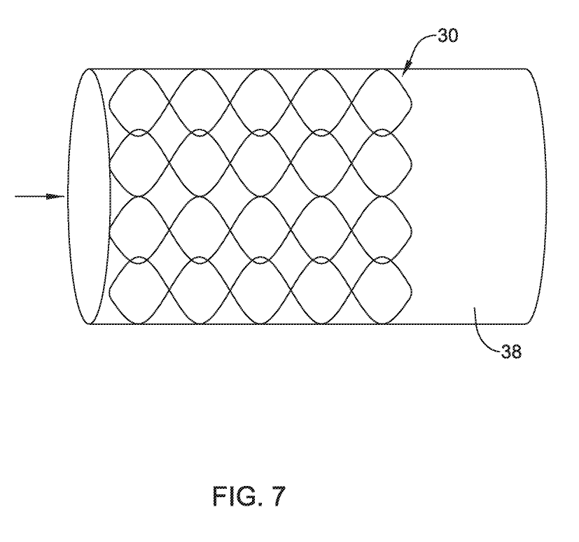

Once planar stent structure 30 has been shaped into a three-dimensional stent design around shaping mandrel 38, it may be removed from shaping mandrel 38 and thereafter resemble the stent structure illustrated in FIG. 1. FIG. 7 illustrates the removal of the stent structure 30 from shaping mandrel 38. For example, FIG. 7 shows an arrow representing the removal of stent structure 30 from mandrel 38.

In some examples, stent structure 30 may undergo a second annealing process prior to the removal from the shaping mandrel 38. For example, while on shaping mandrel 38, stent 30 (shown in FIG. 6), may be undergo a heat set. In some instances this heat set may be a high temperature heat set. Use of the higher temperature heat set may affect the shape memory attributes of the materials used to construct the stent. For example, in some instances, the higher heat set temperature may impart shape memory characteristics into the stent filaments.

As stated, once removed from shaping mandrel 38, stent 10 may resemble the example three-dimensional stent structure shown in FIG. 1. As shown in FIG. 1, the axial bonding region 18 may be defined as including a series of attachment points and/or combined edge regions 36 of the planar stent structure. In some examples, bonding region 18 may resemble that of a seam. In other words, the discrete bonding points may be longitudinally aligned such that they resembled a linear seam along the stent surface. However, in other examples, the discrete bonds 20 of bonding region 18 may not be longitudinally aligned. Rather, it is contemplated that stent 10 may be designed and/or configured such that any portion of filaments 16 may be attached (e.g., welded) to any other portion of filaments 16, irrespective of their linear alignment.

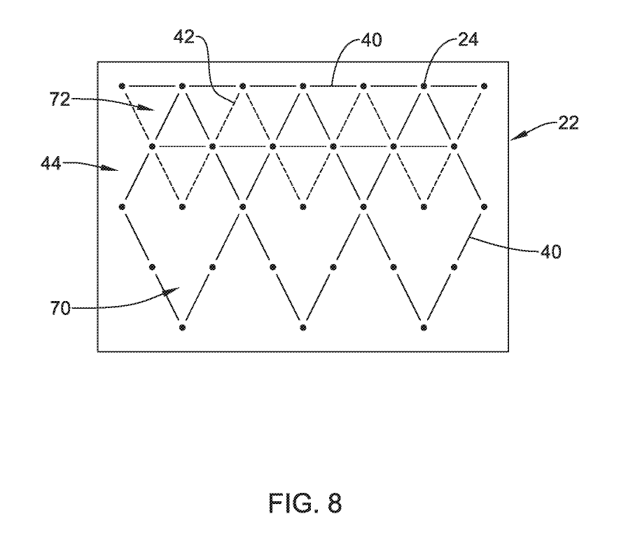

In some instances it may be desirable to utilize one or more different materials to construct the example stent structures disclosed herein. For example, in some instances it may be desirable to incorporate two or more filaments of differing materials when constructing the example stent structures disclosed herein. FIG. 8 illustrates a planar stent structure 44 positioned on a base 22. It can be appreciated that planar stent structure 44 may be formed similarly to the planar stent structure described above in relation to FIG. 4.

However, FIG. 8 further illustrates two different filament materials being utilized to construct structure 44. For example, in some examples a first filament 40 (depicted as a solid line) may be combined (e.g., braided, weaved, knitted, wound, interwoven, etc.) with a second filament 42 (depicted as a dashed line) to form planar stent structure 44. As shown in FIG. 8, filaments 42/44 may be positioned, wound, interwoven, etc. about projections 24. Additionally, as described above with respect to FIG. 4, filaments 42/44 may be interwoven about projections 24 in any given arrangement, pattern and/or distribution. For example, filaments 42/44 may be arranged to form different shapes, spaces, interstices, etc.

For purposes of this disclosure, it is further contemplated that stent structures disclosed herein may be constructed to have interstitial spaces of varying sizes. For example, FIG. 8 shows planar stent structure 44 having an interstitial space 70 that is comparatively larger than interstitial space 72. It can be appreciated that different size stent cells may be formed during the construction of planar stent structures. Further, these relative stent cell sizes may be maintained after an example planar stent structure is subsequently formed into a three-dimensional stent structure as disclosed herein. In some instances, different stent cell openings (e.g., interstitial spaces) may be incorporated into a particular stent design to customize the stent geometry to treat a particular body lumen.

Additionally, different manufacturing methods may be used with a particular material and further combined with different materials and manufacturing methods. For example, in some examples, a first material may be braided and combined with a second material that is knitted. The first and second materials (having been braided and knitted, respectively), may be combined with one another to create a single stent structure. These are just examples. It is contemplated that many different materials may be combined with many different manufacturing methodologies to create both the planar, and subsequently, the three-dimensional stent structures disclosed herein.

While the above example discloses using two different materials to create a planar stent structure, it is not intended to be limiting. For example, it is contemplated that more than two materials may be combined to form the stent structures described herein. For example 2, 3, 4, 5, 6, 7, 8, 9, 10, 15, 20 or more different filament materials may be combined to form the stent structures described herein.

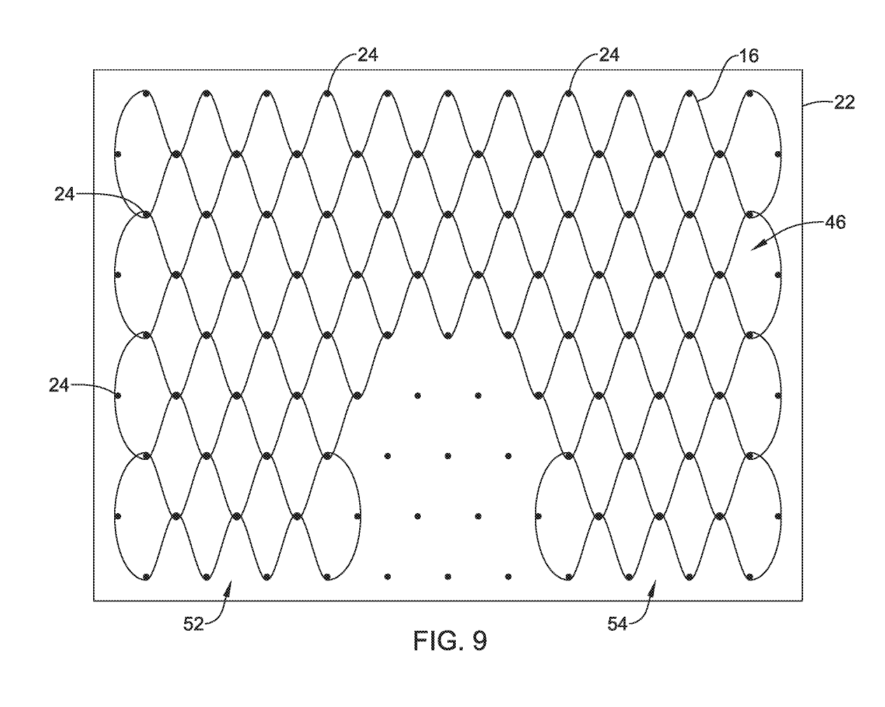

As discussed above, the techniques described herein may be utilized to create varied, complex and/or intricate stent designs and/or configurations. For example, FIG. 9 illustrates an example planar stent pattern 46 designed to form a stent having a bifurcated portion. As shown in FIG. 9, the planar stent pattern 46 may include a body portion 50, a first leg portion 52 and a second leg portion 54. The planar bifurcated stent pattern 46 may be constructed using any of the techniques disclosed herein. For example, the planar bifurcated stent pattern may include one or more filaments 16 positioned (e.g., wrapped, wound, etc.) around projections 24. Filaments 16 may be one or more different materials and interwoven with one another using a variety of manufacturing techniques (e.g., braiding, weaving, knitting, interlocking, interweaving, etc.).

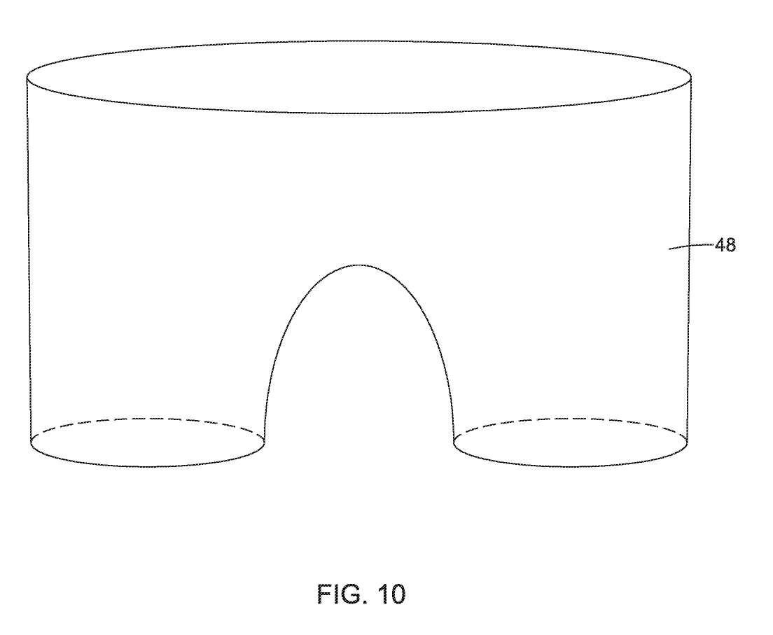

In accordance with some example stent manufacturing methods disclosed herein, the planar bifurcated stent pattern 46 (shown in FIG. 9) may be positioned on a bifurcated shaping mandrel 48 (shown in FIG. 10). While the bifurcated stent pattern 46 is not shown wrapped around bifurcated mandrel 48, it can be appreciated that planar stent 46 may be positioned on mandrel 48 in a similar manner as that described above with respect to FIGS. 4-6.

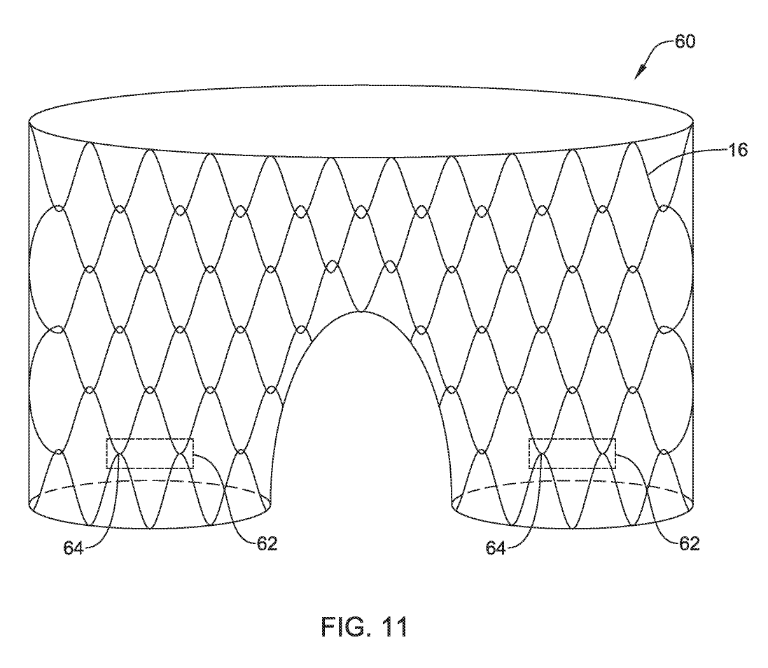

FIG. 11 shows an example bifurcated stent 60 formed in accordance with the methods disclosed herein. For example, bifurcated stent 60 may be defined as the three-dimensional stent structure formed after planar bifurcated stent 46 has been positioned (e.g., wrapped) around shaping mandrel 48 and thereafter removed from shaping mandrel 48.

Additionally, FIG. 11 shows bifurcated stent 60 including one or more axial bonding regions 62 including bonds 64. It is noted that for the purposes of this disclosure, example stent structures formed according to methods disclose herein may include one or more axial bonding regions. For example, stent designs may include 2, 3, 4, 5, 6, 7, 8, 9, 10 or more axial bonding regions. The number and location of a particular axial bonding region within a given stent design may depend on the complexity of a given stent structure.

The materials that can be used for the various components of implantable medical device 10 (and/or other devices disclosed herein) and the various tubular members disclosed herein may include those associated with medical devices. Implantable medical device 10, and/or the components thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 10 to achieve the same result.

In some embodiments, a degree of Magnetic Resonance Imaging (Mill) compatibility is imparted into device 10. For example, device 10, or portions thereof, may be made of a material that does not substantially distort the image and create substantial artifacts (e.g., gaps in the image). Certain ferromagnetic materials, for example, may not be suitable because they may create artifacts in an MRI image. Device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments.

What is claimed is:

1. An implantable endoprosthesis, comprising:
a cylindrical body having a proximal end, a distal end, and an axial bonding region extending between the proximal end and the distal end;
wherein the cylindrical body includes one or more winding filaments; and
a plurality of discrete axial bonds disposed along the axial bonding region, the discrete axial bonds securing together edge regions of the one or more winding filaments.

2. The endoprosthesis of claim 1, wherein the one or more winding filaments includes a braided portion, a knitted portion, or both.

3. The endoprosthesis of claim 2, wherein the one or more winding filaments includes a braided portion and a knitted portion, wherein the braided portion is interwoven with the knitted portion.

4. The endoprosthesis of claim 1, wherein the winding filaments includes a first filament having a first radial compression strength and a second filament having a second radial compression strength different from the first radial compression strength.

5. The endoprosthesis of claim 1, wherein the discrete axial bonds include a weld.

6. The endoprosthesis of claim 1, wherein the cylindrical body further comprises a bifurcated portion.

7. The endoprosthesis of claim 6, wherein the cylindrical body includes a second axial bonding region.

8. An implantable endoprosthesis, comprising:
a tubular scaffold including a proximal end, a distal end and a longitudinal axis, the tubular scaffold including at least a first filament including a first set of windings and a second set of windings;
a bonding region extending along the tubular scaffold including a plurality of discrete bonds;
wherein the one or more discrete bonds secure the first set of windings to the second set of windings.

9. The endoprosthesis of claim 8, further comprising a second filament, wherein the first filament includes a braided portion and the second filament includes a knitted portion.

10. The endoprosthesis of claim 9, wherein the braided portion and the knitted portion are interwoven.

11. The endoprosthesis of claim 8, further comprising a second filament, wherein the first filament includes a first material and the second filament includes a second material different from the first material.

12. The endoprosthesis of claim 8, wherein the tubular scaffold includes a bifurcated portion.

13. The endoprosthesis of claim 12, wherein tubular scaffold includes a second bonding region including a plurality of discrete bonds along the bifurcated portion.

14. A method of making an implantable endoprosthesis, the method comprising:
positioning at least one filament on along a planar surface of a base, the base including a plurality of projections extending away from the surface;
wherein positioning the at least one filament on the planar surface of the base includes winding the at least one filament along the base by winding the filament about the plurality of projections to form a substantially planar stent structure, the planar stent structure including a first side and a second side and one or more interstices therebetween;
removing the planar stent structure from the planar surface;
positioning the planar stent structure around a shaping mandrel; and
attaching the first side of the stent structure to the second side of the stent structure.

15. The method of claim 14, wherein attaching the first side of the stent structure to the second side of the stent structure further includes forming a bonding region.

16. The method of claim 15, wherein the bonding region includes at least one weld.

17. The method of the claim 14 wherein positioning the at least one filament on a planar surface comprises both braiding and knitting the filament around the plurality of projections.

18. The method of claim 14, wherein positioning the planar stent structure around a shaping mandrel includes positioning the planar stent structure around a bifurcated mandrel.

19. The method of claim 14, wherein positioning the at least one filament between at least two of the plurality of projections to form a substantially planar stent structure includes forming a third side and a fourth side.

20. The method of claim 19, further comprising attaching the third side to the fourth side to form a second bonding region.
